(19) United States
(12) Patent Application Publication
(10) Pub. No.: US 2025/0099758 A1
(43) Pub. Date: Mar. 27, 2025

(54) PERIPHERAL NERVE STIMULATION WITH RANDOM VARYING OF FREQUENCY, PULSE AMPLITUDE, DURATION AND/OR PULSE WIDTH FOR IMPROVED PERFORMANCE

(71) Applicant: Kopfwohl GmbH, Stans (CH)

(72) Inventors: Chené Labuschagne, Hermanus (ZA); Hilmar Ehlert, Hergiswil (CH); Jorge Leon Morales Quezada, Belmont, MA (US); Kevin Andrew Duffy, Mortain-Bocage (FR)

(21) Appl. No.: 18/891,019

(22) Filed: Sep. 20, 2024

Related U.S. Application Data
(60) Provisional application No. 63/584,390, filed on Sep. 21, 2023.
(60) Provisional application No. 63/584,400, filed on Sep. 21, 2023.
(60) Provisional application No. 63/584,408, filed on Sep. 21, 2023.
(60) Provisional application No. 63/584,413, filed on Sep. 21, 2023.

(51) Int. Cl. A61N 1/36; A61N 1/04

(52) U.S. Cl. CPC A61N 1/36034; A61N 1/0456

(57) ABSTRACT

Exemplary embodiments may provide systems and methods relating to the treatment of at a subject to enhance performance by the subject. In some exemplary embodiments peripheral nerve stimulation may be used in conjunction along with other treatment options, such as neurofeedback (NFB) and respiratory biofeedback (R-BFB). A subject may be subject in a session of treatment to a period of R-BFB training with peripheral nerve stimulation and a period of NFB training with peripheral nerve stimulation. In other exemplary embodiment the peripheral nerve stimulation is combined with one other type of treatment or with more than two other types of treatment. The treatment may entail multiple sessions over a period of days or even weeks This combined training improves symptoms and may be non-invasive. The frequency of the stimulation, pulse width of the stimulation, duration of the stimulation, or the pulse amplitude of the stimulation may vary during the applying.

### RELATED APPLICATIONS

This application claims the benefit of U.S. Provisional Application No. 63/584,390, filed on Sep. 21, 2023, U.S. Provisional Application No. 63/584,400, filed on Sep. 21, 2023, U.S. Provisional Application No. 63/584,408, filed on Sep. 21, 2023, and U.S. Provisional Application No. 63/584,413, filed on Sep. 21, 2023, the contents of which are incorporated by reference in their entirety.

### BACKGROUND

Neuroplasticity, also known as neural plasticity or brain plasticity, is the ability of neural networks in the brain to change through growth and reorganization. Neuroplasticity refers to the ability of the brain to be rewired to function in some way that differs from how it previously functioned. The rewiring may range from individual neuron pathways making new connections, to systematic adjustments like cortical remapping. Examples of neuroplasticity include circuit and network changes that result from learning a new ability, information acquisition, environmental influences, practice, and psychological stress.

Increasing one's neuroplasticity may help with learning and may result in improved performance in various tasks.

Executive function refers to a set of mental skills that includes working memory, flexible thinking and self-control. When a person has an executive function disorder, the person may have trouble paying attention, remembering things, organizing tasks, and/or managing time. Executive function disorders may be treated with medication and/or therapy, such as cognitive behavioral therapy.

Learning can be difficult for many people. Some people have learning disabilities, whereas other have other learning challenges that make it difficult for them to learn. More generally, there is a demand for treatments that will enhance one's learning.

Given the pace and many challenges of modern life, many people feel anxious and stressed a great deal of the time. While strategies like meditation and exercise can help, for many people those strategies are not enough. As a result, many people rely on medication or alcohol to achieve relaxation and calm. Unfortunately, the medication and alcohol may have side effects and may pose a risk for addiction or overdose.

Because of the pace and challenges people may be preoccupied often or may be concerned with the future. They often are not fully aware of the surroundings and are not appreciating the current moment.

In many endeavors, people are looking to enhance their performance. For example, athletes look to improve their performance in sports. Artists, like musicians, look to improve their artistic performances or works. Similarly, workers seek to improve their performance at work. Currently, the efforts to improve performance are increased largely by practice or training alone.

### SUMMARY

In accordance with an inventive aspect, a method of treating a subject to enhance performance by the subject includes initiating a session of peripheral nerve stimulation with a subject by applying electrical stimulation to a peripheral nerve of the subject. The method also includes randomly varying a frequency, a pulse width, a duration, or a pulse amplitude of the electrical stimulation during at least a portion of the session. The enhancing of performance may enhance, for example, the neuroplasticity, executive function, learning, relaxation, calm, mindfulness, stress control, decision making, motivation, organization, motor skills learning, and pain threshold of the subject.

In some embodiments, multiple ones of the frequency, pulse width, duration and the pulse amplitude of the electrical stimulation may be randomly varied, and the varying may be bound within specified ranges.

In accordance with a further inventive aspect, a method of enhancing performance of a subject is provided. Per the method, a biometric of the subject is measured with a biometric sensor. Biometric feedback is provided to the subject based on the measured biometric, and peripheral nerve stimulation is applied via a peripheral nerve stimulator to the subject as the biometric feedback is provided to the subject for a specified duration. A frequency, pulse width, duration, or pulse amplitude of the stimulation randomly varies during at least a portion of the application.

The biometric sensor may be, for example, a respiratory sensor. In that case, the biofeedback may include an indication of respiration rate and/or respiration pattern or an indication of brain activity of regions of a brain of the subject. A representation of the respiration rate and/or respiration pattern may be displayed on a display of a computing device as part of the biofeedback. The peripheral nerve stimulator may be a median nerve stimulator. The peripheral nerve stimulator may apply an electrical stimulus to the subject. The electrical stimulus may apply a current between 0.25 mA and 10 mA to the subject. The electrical stimulus may have a frequency in the range between 0.25 Hz and 30 KHz, that may change overtime based on the subject's response to the stimulation. The electrical stimulus may be applied for a duration in a range between 1 minute and 50 minutes.

In accordance with another inventive aspect, a method of enhancing performance of a subject entails a respiratory biofeedback (R-BFB), which entails paced breathing training with the subject. Median nerve stimulation is provided to the subject via a median nerve stimulator during the R-BFB training. Sensorimotor rhythm neurofeedback (SMR NFB) training is conducted with the subject, and median nerve stimulation is provided to the subject via a median nerve stimulator during the SMR NFB training. A frequency of the stimulation, a pulse width of the stimulation, a pulse amplitude of the stimulation, or duration of stimulation may randomly vary during at least a portion of at least one of the providing of the median nerve stimulation during the R-BFB paced respiration training or the providing of the peripheral nerve stimulation during the SMR NFB training.

The median nerve stimulator may provide an electrical stimulus. The R-BFB training may be conducted with the subject for a duration between 1 to 5 minutes. The SMR NFB training may be conducted with the subject for a duration between 5 and 30 minutes. The R-BFB-paced respiration training may include providing the subject with feedback regarding a respiration rate of the subject or a respiration pattern of the subject via a computing device. The SMR NFB training may include providing the subject with biofeedback regarding activity of certain brain regions of the subject via a computing device.

In accordance with an additional inventive aspect, a system for treating a subject to enhance performance is provided. The system includes a sensor for sensing a biometric of a subject and a computing device. The computing device includes a storage storing computer programming instructions and a processor configured for executing the computer programming instructions to cause the processor to provide biofeedback training to the subject using the sensed data from the sensor. The system also includes a peripheral nerve stimulator configured to stimulate a peripheral nerve of a subject during the biometric training.

The sensor may include a respiration sensor that senses a respiration rate and/or a respiration pattern of the subject. The sensor may include an electroencephalogram (EEG), a temperature sensor, a heart rate sensor, or a galvanic skin response (GSR) sensor. The peripheral nerve stimulator may be a median nerve stimulator that provides an electrical stimulus. The biofeedback training may be respiratory biofeedback training or sensorimotor rhythm biofeedback training.

In accordance with yet a further inventive aspect, a system for treating a subject to enhance performance is provided. The system includes a peripheral nerve stimulator to provide peripheral nerve stimulation to a subject, the peripheral nerve stimulator being arranged to apply an electrical stimulus to the subject. The electrical stimulus may apply a current between 0.25 mA and 10 mA to the subject. The electrical stimulus may have a frequency in the range between 0.25 Hz and 30 KHz. The electrical stimulus may be applied for a duration in a range between 1 minute and 50 minutes. The frequency of the stimulation, the pulse width of the stimulation, the duration of the stimulation, or the pulse amplitude of the stimulation randomly varies during at least a portion of the applying.

In accordance with yet another inventive aspect, a method of treating a subject to enhance performance includes engaging a subject in a form of treatment to help improve symptoms. The method also includes initiating a session of peripheral nerve stimulation with the subject in conjunction with the treatment by applying electrical stimulation to a peripheral nerve of the subject and randomly varying frequency, pulse width, duration or pulse amplitude of the electrical stimulation during at least a portion of the session.

The treatment may be, for example, one of biofeedback training, neurofeedback training administering medication to the subject, playing a game with the subject, having the subject interact with a computer program, engaging with a therapist, or meditation.

### BRIEF DESCRIPTION OF THE DRAWINGS

FIG. 1 depicts an illustrative system for performing peripheral nerve stimulation in exemplary embodiments.

FIG. 2A depicts an illustrative peripheral nerve stimulator without a sensor for exemplary embodiments.

FIG. 2B depicts an illustrative peripheral nerve stimulator with a sensor for exemplary embodiments.

FIG. 3 depicts the locations of various nerves and arteries in the arm of a subject.

FIG. 4 depicts a block diagram of an illustrative nerve stimulator for exemplary embodiments.

FIG. 5 depicts a diagram of illustrative types of sensors that may be used in the nerve stimulation system of exemplary embodiments.

FIG. 6 depicts in more detail a computing device of the system for performing peripheral nerve stimulation.

FIG. 7A depicts a flowchart of illustrative steps that may be performed in exemplary embodiments in a session where the nerve stimulation is provided in conjunction with both NFB and BFB.

FIG. 7B depicts an illustrative electrical stimulus to aid a patient in sports performance in accordance with exemplary embodiments.

FIG. 7C depicts a flowchart of illustrative steps that may be performed in exemplary embodiments in developing an electrical stimulus to be applied to a subject.

FIG. 7D depicts a pulse description model that describes a pulse by characteristics.

FIG. 7E depicts examples of base pulse descriptions that may be used in exemplary embodiments.

FIG. 7F depicts examples of pattern descriptions that may be used in exemplary embodiments.

FIG. 7G depicts an illustrative stimulus algorithm description that may be used in exemplary embodiments.

FIG. 8 depicts an illustrative arrangement for realizing treatment with the nerve stimulation system and some additional components.

FIG. 9 depicts a flowchart of illustrative steps that may be performed in exemplary embodiments to repeat the training with peripheral nerve stimulation over a period.

FIG. 10A depicts a flowchart of illustrative steps that may be performed in exemplary embodiments where only a single form of treatment is performed with peripheral nerve stimulation.

FIG. 10B depicts examples of treatments that may be performed with a subject in conjunction with peripheral nerve stimulation in exemplary embodiments.

FIG. 11 depicts a flowchart of illustrative steps that may be performed in exemplary embodiments where only peripheral nerve stimulation is performed.

FIG. 12 is a table displaying demographic information and baseline assessment information for participants in a study.

FIG. 13 depicts a table showing a comparison of the ADHD symptoms of study subjects post-treatment versus baseline ADHD symptoms.

FIG. 14 depicts a table showing a comparison of ADHD symptoms of study subjects at follow up versus baseline ADHD symptoms.

FIG. 15 depicts plots of averaged power spectral density (PSD) of basal EEG recordings for baseline and post-treatment and p-values of study subjects in the active group (AG) versus the sham group (SG).

FIG. 16 shows the scalp distribution of the change between baseline and post-treatment evaluation sessions of the Theta-Alpha ratio for the AG versus the SG for study subjects.

FIG. 17 depicts a table of power changes in the Theta band and the Alpha band between baseline and follow-up evaluation for the AG and the SG in the study.

FIG. 18 depicts plots of averaged power spectral density (PSD) of basal EEG recordings for baseline and follow-up and p-values of study subjects in the AGversus the SG.

FIG. 19 shows the scalp distribution of the change between baseline and follow up evaluation sessions of the Theta-Alpha ratio for the AG versus the SG for study subjects.

FIG. 20 depicts a table that shows the correlation of the power changes in Theta and Alpha bands with the clinical changes for each variable in AG and SG, calculated with Pearson's method, for baseline and post-treatment.

FIG. 21 depicts a table that shows the correlation of the power changes in Theta and Alpha bands with the clinical changes for each variable in AG and SG, calculated with Pearson's method, for baseline and follow up.

### DETAILED DESCRIPTION

Exemplary embodiments may provide systems and methods relating to the treatment of a subject to enhance performance. The exemplary embodiments, for example, may enhance the neuroplasticity, executive function, learning, relaxation, calm, mindfulness, stress control, decision making, motivation, organization, motor skills learning, and pain threshold of the subject.

In some exemplary embodiments peripheral nerve stimulation alone may be used for treatment. The stimulation pattern may be chosen to have an effect that is beneficial to the patient. The stimulation pattern may be characterized by duration, pulse amplitude, pulse width, frequency, etc. The stimulation provided by a peripheral nerve stimulator may randomly vary in frequency, pulse amplitude, duration and/or pulse width over at least a portion of the stimulation. The variance in frequency, pulse amplitude, duration and/or pulse width may be limited to vary within particular ranges. This random variance in frequency, pulse amplitude, duration and/or pulse width may help avoid neuro-habituation of the subject being stimulated so that the subject continues to respond well to the stimulation after repeated stimulation and to yield a better overall result relative to a fixed stimulation pattern. The frequencies, pulse amplitudes, durations and/or pulse widths may be randomly chosen by one or more pseudo-random computing functions or other methods of creating random selections. The random modulation of the frequency is also sometimes referred to as adding of random noise, and in some embodiments wider frequency ranges up to 30 kHz can be used.

In other exemplary embodiments peripheral nerve stimulation may be used in conjunction with other treatment options, such as neurofeedback (NFB) and respiratory biofeedback (R-BFB). For example, a subject may be subject in a session of treatment to a period of R-BFB training with peripheral nerve stimulation and a period of NFB training with peripheral nerve stimulation. In yet other exemplary embodiments, the peripheral nerve stimulation is combined with one other type of treatment or with more than two other types of treatment. For example, the peripheral nerve stimulation may be used with a treatment by medication in some instances. The peripheral nerve stimulation may accompany other forms of treatment, such as a game, an interaction with a computer program, meditation, therapy with a therapist or the like. The treatment may entail a single session or multiple sessions over a period of days or even weeks. This combined training may improve the condition and/or symptoms, without using medication or may be combined with a reduced dose of medication. The neural stimulation may be non-invasive.

The peripheral nerve stimulation may stimulate the median nerve in some exemplary embodiments. The peripheral nerve stimulation may stimulate the ulnar nerve in other exemplary embodiments, and in some exemplary embodiments, the peripheral nerve stimulation stimulates both the median and ulnar nerves. The median nerve may be stimulated by a nerve stimulator positioned, for example on skin located on the hand, arm or wrist of a subject. In some exemplary embodiments, the stimulation may be realized by electrodes of a nerve stimulator that apply an electrical stimulus. In other exemplary embodiments, the stimulus may be vibrations, magnetic fields, light, sound and/or pressure.

During peripheral nerve stimulation the electrical impulses follow the peripheral nerve pathway reaching the ascending reticular formation in the brain stem, increasing the excitability in this area and modulating the sensori-motor thalamocortical pathways (M1 and S1 cortices), as well as the insula and other cortical structures. Nerve stimulation in this manner thus represents an affordable and non-invasive technique that has the potential to induce metaplasticity within the somatosensory networks via the spinothalamic tract.

Combining peripheral techniques with bottom down anxiety regulation techniques is believed to enhance this proven technique by adding bottom up regulation, the stimulation of the sensory motor system (M1/S1) facilitates the co-activation of networks related to anxiety relief networks, specially by facilitating SII activation.

Cognitive training is directly targeted at functions such as working-memory, executive control and attention. Encouraging data on its effects on cognitive performance, self-efficacy and everyday functioning have boosted its diffusion in healthy populations across the lifespan, as well as in psychiatric and neurological conditions, particularly when pharmacological support is limited or absent. A key criterion for assessing the outcome of any training is the generalization of acquired skills to non-trained functions, that is, so-called “transfer” of function. Despite encouraging evidence of transfer to non-trained cognitive functions and long-lasting effects, the actual extent of cognitive training effects is debated. Median nerve stimulation (MNS) might help to sustain the effects from cognitive/executive training, by promoting mechanisms associated with synaptic plasticity. Combining cognitive and physical interventions with neuromodulation techniques is promising because of the larger effects of dual-task training compared with either modality alone, both in healthy subjects and patients. While generally used for motor or cognitive rehabilitation, indeed, physical interventions have also been shown to improve cognitive performance, by targeting either the intensity of training (aerobics and strength), or its complexity (balance, coordination and flexibility). Their effects might be mediated by the resulting increase of brain plasticity which is boosted by the role of MNS during training, resulting in enhanced executive functions and working-memory, and decreasing the risk of cognitive decline. Cognitive and physical training plus MNS seem to induce complementary effects at the neural level, that is, maintaining functional response versus structural/volumetric changes, respectively.

Peripheral nerve stimulation can facilitate learning by modulating inputs from sensory and electrical stimulation. These circuits engage the sensorimotor system (M1/S1 cortex) serving as a primer for supplementary co-activation of distant networks such as attention and WM networks located in prefrontal and temporal areas respectively. If subject is exposed to executive tasks, (for example forward-digit span tasks and Corsi Block test for WM; verbal fluency, and category or semantic fluency for cognitive flexibility; Stroop test for inhibition tasks) with peripheral nerve stimulation, the effects of such activities may be enhanced by promoting processes associated with neuroplasticity by cortical co-activation processes.

Peripheral nerve stimulation may be used as an adjuvant technique to facilitate executive functions performance to enhance learning and consolidate memory processes by promoting coactivation of different brain areas. Stimulation parameters (intensity, frequency, duration, or parameters that affect the shape of the pulse) and cognitive tasks metrics (reaction time, errors, commissions, omissions) may be used to individualize user experience, by collecting and analyzing on-line/stored data for algorithm generation.

When a person is subject to stress, their body releases hormones that raise their blood pressure. When a person is subject to repeated stress, their blood pressure rises repeatedly and may result in hypertension (along with other factors). Peripheral nerve stimulation may help to modulate the autonomic nervous system so as to help modulate the response to stress and decrease blood pressure. The peripheral nerve stimulation may also help modulate levels of orexin, a hormone involved in stress response. Moreover, the peripheral nerve stimulation with biofeedback training may help bring about feelings of wellness and calm that help to decrease stress responses.

One way in which performance may be enhanced for an athlete is by raising the pain threshold of the athlete. Some of the areas activated by sensorial stimulation, namely, primary and secondary somatosensory, as well as the insula, overlap with the ones though to represent the pain matrix—a network that has been shown to be involved in the processing of nociceptive stimuli. Therefore, by a co-activation mechanism, the sensorial stimulation may have an impact in pain processing. As such, sensorial stimulation may be used in order to increase pain threshold, which can be useful in clinical rehabilitation; or in other areas where pain plays a role, such as sports endurance, where athletes have been shown to have increased pain threshold, when compared to non-athletes.

In addition, peripheral nerve stimulation with practice-related training may enhance motor skills learning for athletic endeavors. Peripheral nerve stimulation may boost neuroplasticity and enhance learning.

Peripheral nerve stimulation and training may also help performance at work or in other endeavors. Peripheral nerve stimulation and training may enhance stress control, decision making, motivation and organization. Hence, performance of such endeavors may be enhanced.

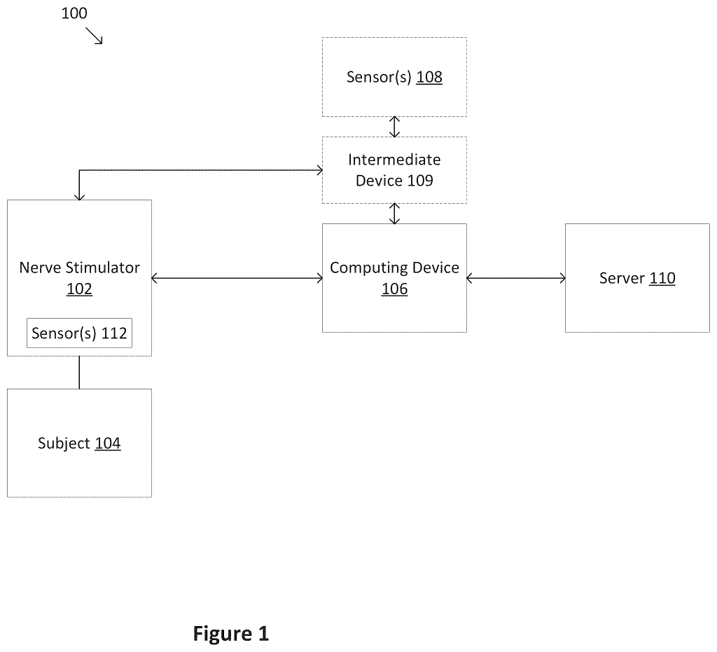

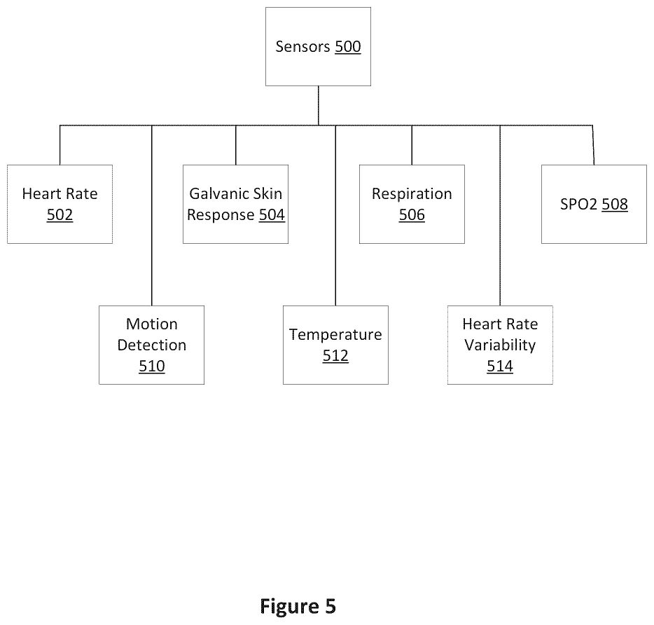

FIG. 1 depicts an illustrative system 100 for performing peripheral nerve stimulation in exemplary embodiments. The system may include a nerve stimulator 102 for stimulating at least one peripheral nerve of a subject 104. The nerve stimulator 102 may be worn or positioned on the subject 104 or may be arranged in proximity to the subject 104. The nerve stimulator 102 may provide a stimulus to the targeted peripheral nerve(s), such as the median nerve or ulnar nerve. The stimulus may be electricity, sound, light, a magnetic field, vibrations, pressure, etc. For a light stimulus, a light source, such as a light emitting diode (LED) or a laser, may direct light to stimulate the peripheral nerve(s). An illustrative embodiment is described below that deploys electrical stimulation. For an electrical stimulus, an electrical current is applied to the peripheral nerve(s). For a sound stimulus, a sound generator may generate sound waves that are directed to stimulate the peripheral nerve(s). For a magnetic field stimulus, a time-varying magnetic field generator may direct a magnetic field to induce an electrical current in the subject's tissue to stimulate the peripheral nerve(s). For a vibration stimulus, a vibration generator may generate vibrations to stimulate the peripheral nerve(s). For a pressure stimulus, pressure may be generated to be applied to stimulate the peripheral nerve(s). The nerve stimulator 102 may include one or more integrated sensors 112 in some embodiments or may include no integrated sensors in other embodiments. The integrated sensor(s) 112 may be biometric sensors or other types of sensors, like those identified in FIG. 5 and described below.

The nerve stimulator 102 may communicate with a computing device 106. The computing device 106 may be a personal computer, a laptop computer, a table computer, a smartphone, a server computer or other variety of computing device 106. The computing device 106 may receive data from the nerve stimulator 102 and send data to the nerve stimulator 102. The computing device 106 may include software for performing NFB, BFB, or other types of treatment like engaging the subject in a game or with an interactive computer program. An intermediate device 109, such as another wearable device that interfaces with the sensor(s) 108, may be provided. The intermediate device 109 may receive sensor data from the sensor(s) 108, forward the sensor data to the nerve stimulator 102 and/or may process or store sensor data. The intermediate device 109 also may control the nerve stimulator 102 via commands or other communications. Examples of the intermediate device 109 include a smartwatch, a fitness tracker, a wearable health monitor, a sleep analyzer, or the like. The sensor data may be used in the training and in closed loop control of the nerve stimulation as is described below. The intermediate device 109 may also pass sensor data to the computing device 106 for analysis, display or storage in some instances. A server computing device 110 may be provided. The server computing device 110 may, in some exemplary embodiments, be part of a cloud computing environment or cluster. Alternatively, the server computing device 110 may be a standalone server with a network connection to the computing device 106. The server computing device 106 may, in some embodiments, include a tool for creating and storing stimulation algorithm descriptions that detail the stimulus to be applied to a subject. The algorithms may be forwarded to the nerve stimulator to be stored therein and applied by the nerve stimulator as required.

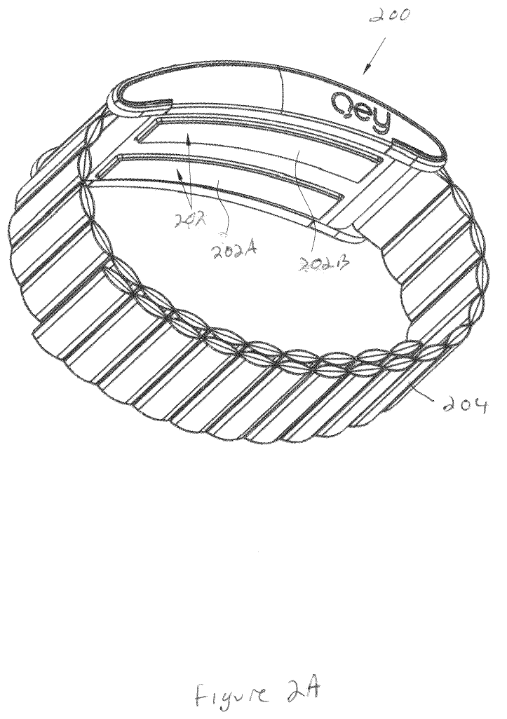

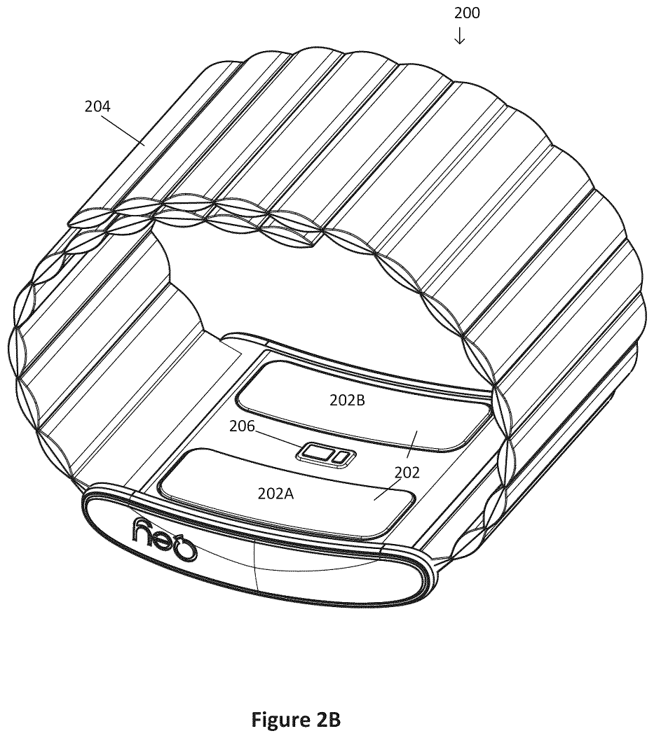

FIG. 2A depicts an illustrative nerve stimulator 200. The nerve stimulator 200 may include electrodes 202 positioned to deliver an electrical output to stimulate a median nerve or ulnar nerve of the subject with an electrical stimulus. In the depicted embodiment, the nerve stimulator includes electrodes 202A and 202B. The electrodes 202 may apply the electrical stimulus to the subject's skin, proximate to the anterior or ventral side of the subject's arm, wrist or hand. The nerve stimulator 200 may take the form of a portable, wearable stimulator, which may be embodied and/or housed and/or accommodated and/or integrated within and/or secured to (with a clip, for example) a fitness tracker band, clasp, patch, strap, or sleeve or conventional watch. In one version, and with particular reference to watches (typically, upper end, luxury watches) that make use of so-called butterfly clasps comprising a central, curved clipping body, to which adjacent flexible strap portions are hingedly fitted, the nerve stimulator may be integrally fitted, or removably fittable, to the clipping body. In some exemplary embodiments the nerve stimulator 200 may be integrated with a smartwatch. The smartwatch may include a watch computing device fitted to a strap 204, such as on the dorsal side of the subject's wrist, with the ventral side of the strap comprising the nerve stimulator 200. The nerve stimulator 200′ shown in FIG. 2B depicts a nerve stimulator like that shown in FIG. 2A but with an integrated sensor 206. The nerve stimulator 200′ includes electrodes 200, which include electrode 202A and electrode 202B. The nerve stimulator 200 may include a computing device fitted to a strap 204 as in FIG. 2A.

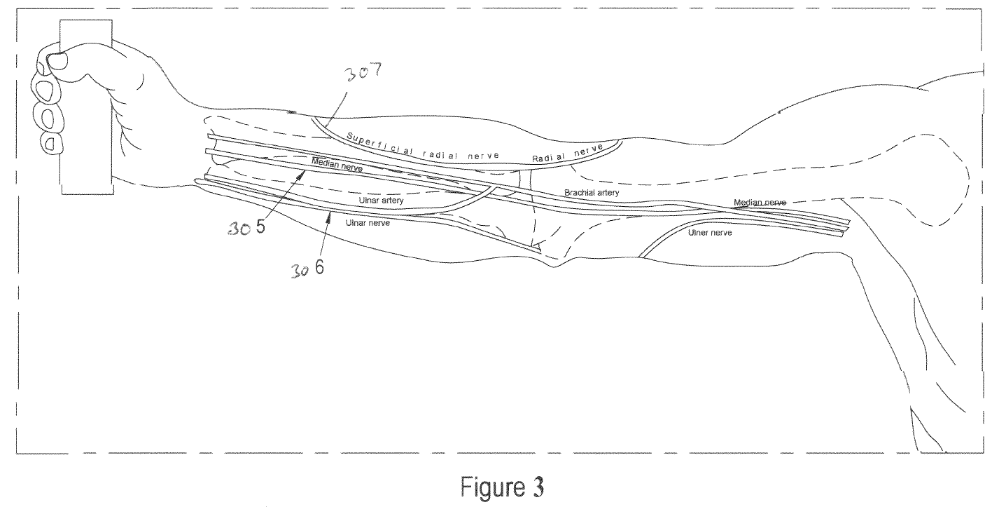

FIG. 3 depicts various nerves and arteries in the arm. The median nerve 305, the ulnar nerve 306, and the radial nerve 307 are all depicted. The electrodes 202 may stimulate the median nerve 305 and ulnar nerve 306 given the path that the nerves follow in the arm. The radial nerve 307 may require that the nerve stimulator be positioned on the arm rather than the wrist. The median 305, ulnar 306, and radial 307 nerves form conduits to the brain and autonomic nervous system via the brachial plexus and spinal nerves. Stimulation of these nerves 305, 306, and/or 307 can influence motor/sensory brain functions, as well as the autonomic nervous system.

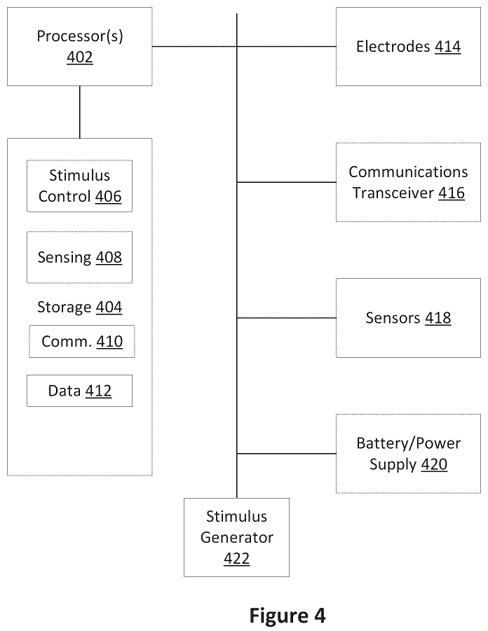

FIG. 4 depicts a block diagram of an illustrative nerve stimulator 400. The nerve stimulator 400 includes one or more processor(s) 402. The processor(s) 402 may include central processing units (CPUs), graphics processing units (GPUs), application specific integrated circuits (ASICs), field programmable gate arrays (FPGAs), and/or other electrical circuitry. The processor(s) 402 may execute computer programming instructions to control operation of the nerve stimulator 400. Each processor may include one or more cores.

A storage 404 may be provided in the nerve stimulator 400. The storage 404 may include one or more kinds of memory and/or storage devices. The storage 404 may include random access memory (RAM), read only memory (ROM), solid state storage, magnetic storage devices, optical storage devices and other types of non-transitory computer-readable storage. The storage 404 may store computer program instructions that may be executed by the processor(s) 402. These computer programming instructions may include computer programming instructions for controlling delivery of the stimulus to the subject 406. These instructions may dictate when a stimulus is delivered and may control the particulars of the stimulus like frequency, amplitude, duration, pulse length, etc. Computer program instructions 408 for receiving and processing sensor data may be stored in the storage 404. The storage 404 also may store instructions 410 for realizing communications with other devices and for interfacing with the communications transceiver 416. The storage 404 may also store data 412, such as stimulation profiles received from the server computing device 106 for subjects and other data. The data 412 may include sensor data that may be used in a closed loop arrangement to influence the stimulus control 406 of the nerve stimulator 400.

The nerve stimulator 400 may include electrodes 414. The electrodes 414 may be used to deliver electrical stimuli to subjects. The nerve stimulator 400 may include a communications transceiver 416. In some exemplary embodiments, the communications transceiver 416 may facilitate wireless communications with other devices, including the computing device 106 and the intermediate device 109. The communications transceiver 416 may enable communications via wireless protocols such as Bluetooth, Bluetooth Low Energy (BLE), near field communications (NFC), etc. Sensors 418 may be integrated into the nerve stimulator in some exemplary embodiments as was mentioned above. This integration of the sensor(s) into the nerve stimulator 400 may help realize more efficient and possibly faster operation since the sensor data need not be obtained from external sensor(s).

FIG. 5 depicts a diagram of illustrative types of sensors 500 that may be used in the nerve stimulation system 100. The sensors 500 may be integrated into the nerve stimulation device 400 or may be separate as part of the sensor(s) 108. A heart rate sensor 502 may be provided to monitor the heartrate of the subject. A galvanic skin response (GSR) sensor 504 may be provided to monitor perspiration of the subject. The GSR may be used, for example, to adjust pulse amplitude to account for different contact resistance due to skin types, humidity, etc, A respiration sensor 506 may be provided to monitor the rate of respiration of the subject. An oxygen saturation sensor 508 (SPO2) may be provided to sense the oxygen saturation level in the blood of the subject. A motion detection sensor 510, such as a gyroscope, may be provided. A temperature sensor 512 may be provided for sensing the temperature of the subject. A heart rate variability sensor 514 may be provided to sense variability in the heart rate of the subject. This enumeration of sensors is not intended to be exhaustive. Other types of devices, like a global positioning system (GPS) device may be used as well.

The nerve stimulator 400 may also include a battery or power supply 420 that powers the nerve stimulator and provides the electrical source for the electrical stimulus. The battery or power supply may be realized as one or more button cell batteries in some exemplary embodiments and may be rechargeable. The nerve stimulator 400 may also include a stimulus generator 422 for generating the electrical stimulus. The stimulus generator 422 may include electrical components for generating the stimulus from the battery/power supply 420. The stimulus generator 422 may be under the control of the processor(s), which instruct the stimulus generator 422 to generate a stimulus with particular characteristics (e.g., frequency, amplitude, duration, pulse width, etc.).

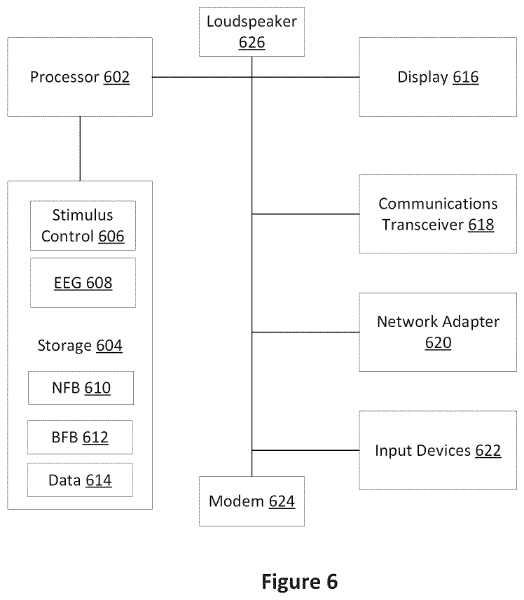

FIG. 6 depicts the computing device 600 in more detail. The computing device 600 includes a processor 602. The processor 602 is configured to execute computer programming instructions to control operation of the computing device 600. The processor 602 may include one or more CPUs, GPUs, ASICs, FPGAs or other types of processing circuits. The processor 602 may have multiple cores. The computing device 600 may include a storage 604 for storing computer programming instructions, data, documents, etc. The storage 604 may include non-transitory computer-readable storage and memory devices like those discussed above relative to storage 404. The storage 604 may store computer programming instructions 606 for generating and controlling the stimulus for the subject. The storage 604 may store computer programming instructions 608 for controlling and gathering electroencephalogram (EEG) data from the subject. The storage 604 may store computer programming instructions 610 for facilitating the gathering of data and the use of NFB and computer programming instructions 612 for facilitating the gathering of data and use of BFB. The storage 604 may also store data 614.

The computing device 600 may further include a display 616 for displaying textual, graphical and other content. The display 616 may be a liquid crystal display (LCD), a light emitting diode (LED) display, a retinal display, a plasma display, a cathode ray tube (CRT) display, an organic LED (OLED) display, a touchscreen display, or the like. The computing device 600 may include a communications transceiver 618 for receiving and transmitting of wireless messages via a wireless protocol like Bluetooth, BLE, NFC, etc. The computing device 600 may include a network adapter 620 for interfacing with a local area network, such as a WiFi network, an Ethernet network, or other variety of network. The computing device 600 may include input devices 622, such as a keyboard, a mouse, a thumbpad, a microphone, a touchscreen, etc. for providing input to the computing device 600. The computing device 600 may include a modem 624, such as a cellular modem. The computing device 600 may include a loudspeaker 626 for providing audio output.

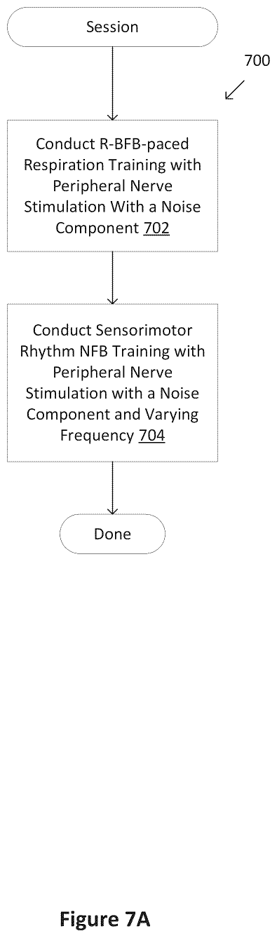

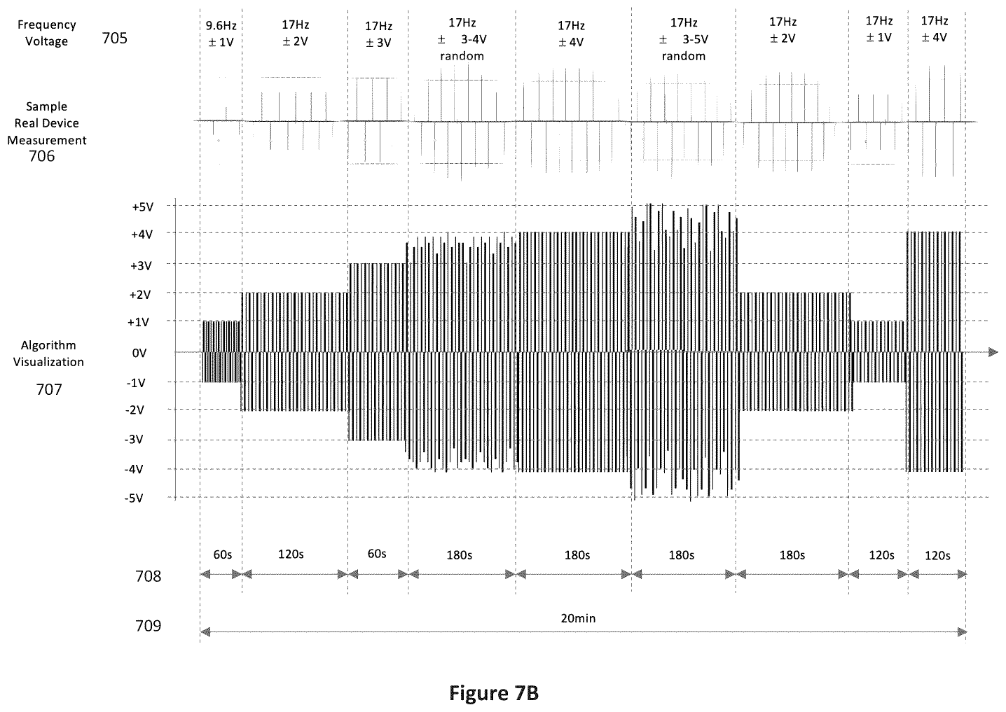

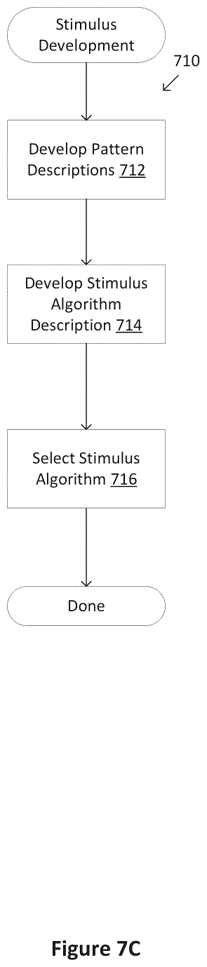

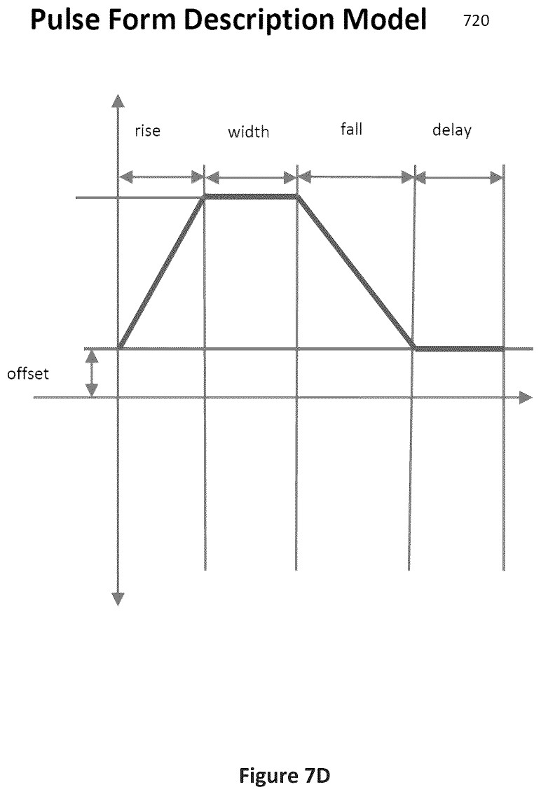

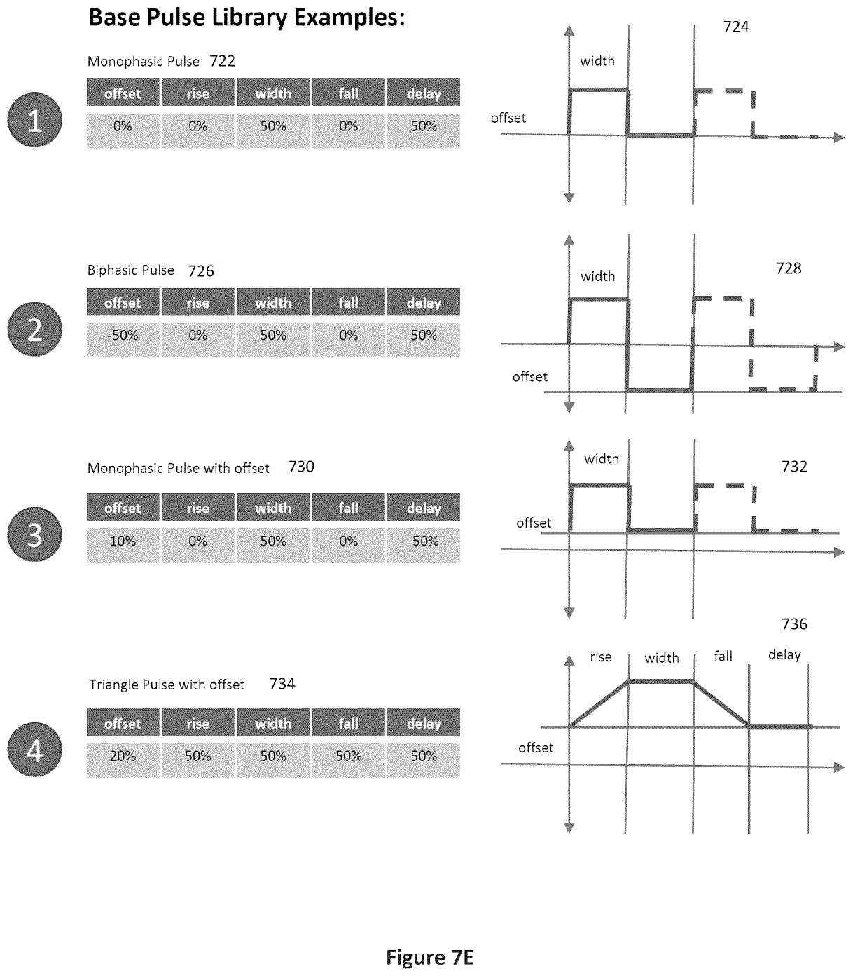

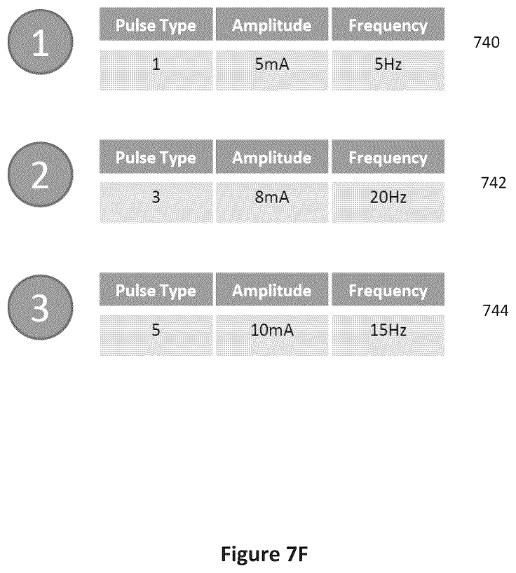

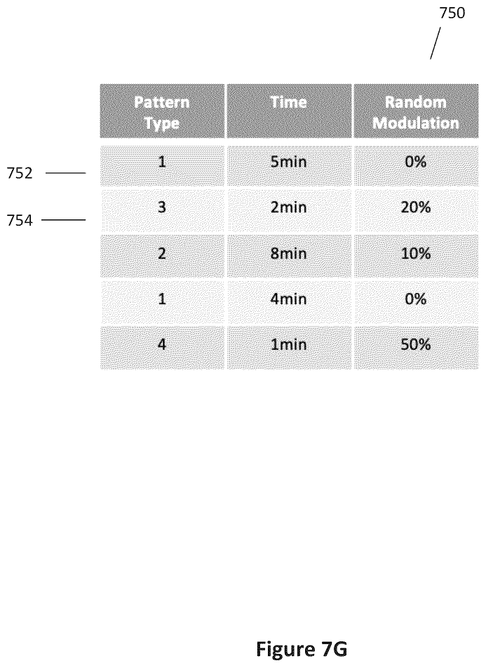

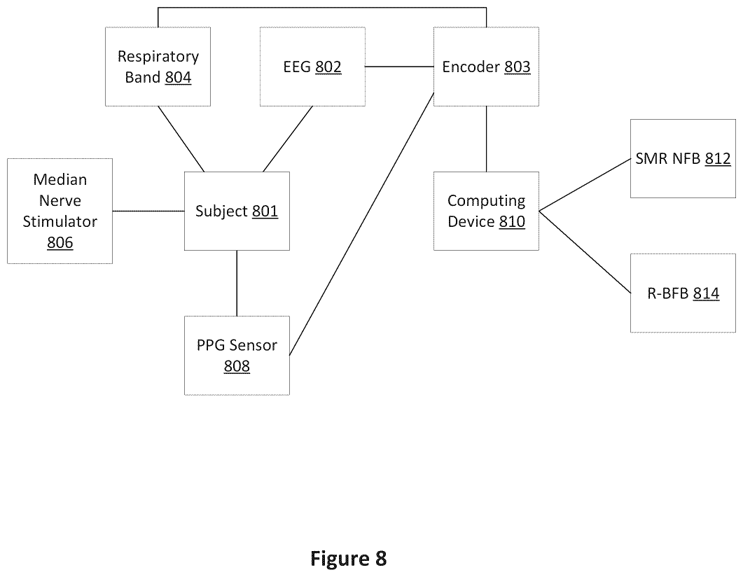

In some exemplary embodiments, the nerve stimulator 400 is adapted to operate in conjunction with either a NFB and/or a BFB therapy arrangement, which forms part of a brain/body-computer interface (BBCI). The treatment may be provided in a series of treatment sessions. FIG. 7A depicts a flowchart 700 of illustrative steps that may be performed in exemplary embodiments in a treatment session where the nerve stimulation is provided in conjunction with both NFB and BFB. FIG. 8 depicts an illustrative arrangement 800 for realizing this treatment of a subject 801 with the previously described nerve stimulation system and some additional components. At 702, R-BFB paced respiration training may be performed along with peripheral nerve stimulation. The R-BFB arrangement includes a respiratory band 804 to monitor, measure and record breathing rates and patterns. The data generated by the respiratory band 804 is encoded by encoder 803 (which may be realized as software and hardware of the intermediate device 109) responsive to the breathing of the subject 801 is received, stored and processed by the computing device 810. These metrics are converted into real-time visual and auditory feedback 814, which is then presented to the subject 801 via the display and the loudspeaker of the computing device 810. This process empowers individuals to engage in self-regulation, allowing them to modulate the activity of specific brain regions linked to behaviors or symptoms in a personalized manner. A photoplethysmography (PPG) sensor 808 may be provided to gather heart rate data.

The peripheral nerve stimulation may facilitate adequate behavioral responses by modulating thalamo-cortical inhibitory inputs with the electrical stimulation. These circuits may engage the sensorimotor system (M1/S1 cortex) serving as a primer for supplementary co-activation of distant networks when a subject is being exposed to cognitive or physical tasks. Moreover, if targeted training is introduced, peripheral nerve stimulation seems to enhance the effects of such activity by promoting processes associated with neuroplasticity. Overall, following this rationale, peripheral nerve stimulation may be used as an adjuvant technique to facilitate inhibitory circuits and enhance attention function in patients.

The nerve stimulation may be delivered as a current between 0.25 mA to 10 mA, such as 2 mA. The frequency of the stimulus may be a random frequency in the range of 0.25 Hz to 30 KHz, such as in the range of 2 Hz to 15 Hz or in the range of 4 Hz to 10 Hz. The stimulus may be delivered via the nerve stimulator, such as median nerve stimulation (MNS) device 806 for a duration of 1 to 50 minutes, such as a duration between 2 to 8 minutes, like a 5 minutes duration. The electrical impulses of the stimulus follow the median nerve pathway of the subject 801 and reach the ascending reticular formation in the brain stem, increasing the excitability in this area and modulating the sensori-motor thalamocortical pathways (M1 and S1 cortices), as well as the insula and other cortical structures. Similar MNS may be applied to enhance executive function and to enhance learning.

FIG. 7B depicts a visualization of the stimulus for an illustrative algorithm where the stimulation is for improving sports performance but a similar pattern may be used for treating ADHD. The pattern may vary for other applications and the depicted pattern is intended merely to be illustrative. The stimulation algorithm has 9 phases of varying length. As can be seen in timeline 708, the first phase is 60 seconds long, whereas the second phase is 120 seconds long. As indicated by timeline 709, the entire session lasts 20 minutes. As can be seen at 705, the frequency of the stimulus may vary among phases. For example, the frequency is 9.6 Hz for the first phase and 17 Hz for the second phase. The voltage (i.e., the pulse amplitude) is plus or minus 1 volt for the first phase and plus or minus 2 volts for the second phase (see 705). This can be seen in the algorithm visualization 707 for these phases. Sample real device measurements 706 are shown as well.

As mentioned above, the frequency, pulse amplitude, duration, and pulse width may randomly vary. As can be seen for the fourth phase, the amplitude randomly varies within a specified range between +3 volts and +4 volts and −3 volts and −4 volts. Such random variance in pulse amplitude also occurs in the sixth phase. The pulse width may randomly vary as well but is not immediately discernable from the visualization 707.

The varying in the frequency, pulse width, pulse amplitude, and duration may help to prevent neuro-habituation, where the response to a same stimulus becomes lessened over time due to the subject becoming habituated to the stimulus. The frequencies, pulse widths, pulse amplitudes, and durations may be randomly chosen by a pseudo-random computing function or other means.

FIG. 7C depicts a flowchart 710 of illustrative steps that may be performed in exemplary embodiments in developing an electrical stimulus to be applied to a subject. A pattern description may be developed at 712. The pattern description specifies a pulse type with parameters, such as amplitude, pulse width, and frequency. The pulse type specifies a type of pulse among those in a base pulse library that may be stored in the storage 404 of the peripheral nerve stimulator 400. FIG. 7D depicts a pulse description model 720 that describes a pulse by characteristics like offset, rise, width, fall and delay. FIG. 7E gives examples of base pulse descriptions that may be stored in the storage 404. For each pulse type, there is a table. In this example, there is a table 722 for a monophasic pulse, a table 726 for a biphasic pulse, a table 730 for a monophasic pulse with an offset, and a table 734 for a triangle pulse with an offset. Each of these tables 722, 726, 730, and 734 includes a specification for the offset, rise, width, fall, and delay for the associated pulse type per the pulse form description model of FIG. 7D. FIG. 7E also depicts graphic examples 724, 728, 732, and 736 of the pulse types. These examples need not be stored in the base pulse library and are merely illustrative of how the associated pulses are shaped. The pulse types are intended to be merely illustrative. Other pulse types may be specified.

FIG. 7F depicts examples of pattern descriptions 740, 742, and 744. Each example pattern description 740, 742, and 744 specifies a pulse type, an amplitude, and a frequency. For example, pattern description 740 specifies pulse type 1 from the base pulse library. The pulse has an amplitude of 5 mA and a frequency of 5 Hz. In contrast pattern description 742, specifies pulse type 3, an amplitude of 8 mA, and a frequency of 20 Hz. These are examples of the pattern descriptions developed at 712 of FIG. 7C.

At 714 of FIG. 7C, the stimulus algorithm description is developed. The pattern descriptions are referenced in the stimulus algorithm description. FIG. 7G depicts an illustrative stimulus algorithm description 750. The stimulus algorithm description 750 specifies a sequence of patterns that are applied sequentially in the stimulus algorithm. For instance, row 752 specifies the pattern that is applied first, Row 754 specifies the pattern that is applied second and so forth. Row 752 specifies that pattern type 1 is applied. The duration that the pattern to be applied is also specified. For row 752, the duration is specified as 5 minutes. Each row also specifies how much, if any, random amplitude modulation is applied to the pattern. For row 752, no random noise is applied. In contrast for row 754, 20% random modulation is applied. The percentage specifies the range in which the amplitude of the pulses may vary. For instance a 20% modulation for a pulse with a target amplitude of ±5 volts, implies that the amplitudes may be modulated to fall within ±5 volts to ±6 volts.

As can be seen from the example algorithm description table 750 of FIG. 7G, each pattern may vary as to pattern type, duration, and magnitude of random modulation. Thus, the algorithm description 750 may specify changes in pulse patterns, durations and magnitude of random modulation over the course of a session.

Once an algorithm description has been developed, it may be selected and applied to a subject at 716. In some exemplary embodiments, the algorithm description may be developed and stored on a server 110 or other computing device 106 and forwarded from the server 110 to a local computing device 106 and then onto the peripheral nerve stimulator 102 for application to the subject. In some exemplary embodiments, a calendar schedule may be provided. Activation times of the algorithms may be reflected in the calendar schedule, and the calendar schedule may be used by the neural stimulator to activate the algorithms at the scheduled times. Alternately, the wearable nerve stimulator, such as depicted in FIGS. 2A and 2B may include a button or other device for manually activating a stimulation. Still further the scheduling and activation may be controlled by computer programming instructions, such as the stimulus control 406 instructions.

This R-BFB training with peripheral nerve stimulation (702) may be followed at 704 with sensorimotor rhythm (SMR) NFB training with peripheral nerve stimulation by the wearable MNS device 806.

An EEG sensor 802 may be used in the NFB therapy to register the EEG of the subject. The EEG data may be encoded by encoder 803 and the encoded data may be processed by the computing device to calculate different metrics from this signal, such as power spectrum, connectivity and entropy. These metrics may be converted into real-time visual and auditory feedback 812, which may then be presented to the subject 801. This process may empower the subject 801 to engage in self-regulation to modulate the activity of specific brain regions linked to behaviors or symptoms in a personalized manner.

The stimulus may be delivered during the SMR NFB training as a current in the range of 0.25 mA to 10 mA, such as at a current of 2 mA. The frequency of the stimulus may be at a random frequency in the range of 0.25 Hz to 30 KHz, such in a subrange of 10 Hz to 20 Hz or a subrange of 12 Hz to 16 Hz. The duration of the stimulus may be between 1 minute and 50 minutes, or in a subrange of 10 minutes to 30 minutes, such as 20 minutes.

A low frequency stimulation in range of 5 Hz to 8 HZ may be used with the R-BFB paced respiration training to induce a state of relaxation in some embodiments. This may be followed by a stimulation with an oscillating frequency ranging between 10 Hz and 15 Hz during the SMR NFB training, which is believed to increase sustained attention in some embodiments.

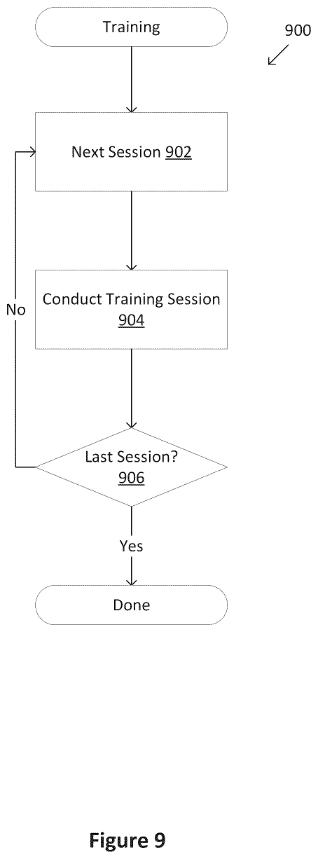

The training of the subject 801 may be repeated in sessions over a series of days, weeks, or months. FIG. 9 depicts a flowchart of illustrative steps that may be performed in exemplary embodiments to repeat such training over a period. A next session is initiated at 902. At 904, the training as detailed above relative to FIGS. 8 and 9 is performed. At 906, a check is made whether the session was the last session of training. If so, at 906, the training is done, if not the next session is initiated at a scheduled date and time. It should be appreciated that in some instances, the training may be ongoing.

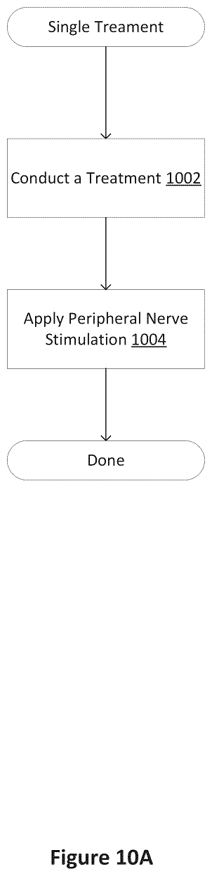

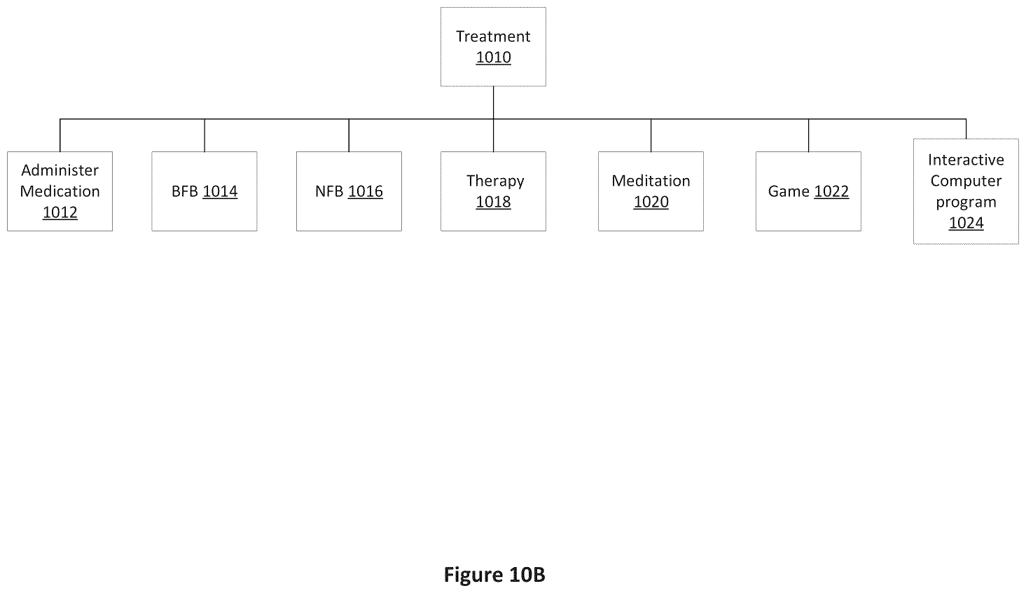

In some exemplary embodiments, only a single treatment is applied to the subject in conjunction with the peripheral nerve stimulation to treat a subject to enhance performance by the subject. FIG. 10A depicts a flowchart 1000 for such a case. The treatment 1010 may take many forms as shown in FIG. 10B. For instance, the treatment 1010, may include the administering of medication 1012 to the subject to treat the conditions and/or symptoms. The treatment 1010 may include BFB 1014 or NFB 1016 as described above The treatment 1010 may include therapy 1018 with a therapist, such as cognitive, behavioral therapy or psychotherapy. The treatment 1010 may include meditation 1020. The treatment 1010 may include having the subject engage with a game 1022. The treatment 1010 may include the subject interacting with an interactive computer program 1024. Other forms of treatment may be used as well.

In conjunction with the treatment, at 1004, peripheral nerve stimulation be applied to the subject. The peripheral nerve stimulation may include random variance in frequency, pulse amplitude, duration and/or pulse width during at least a portion of the applying of MNS.

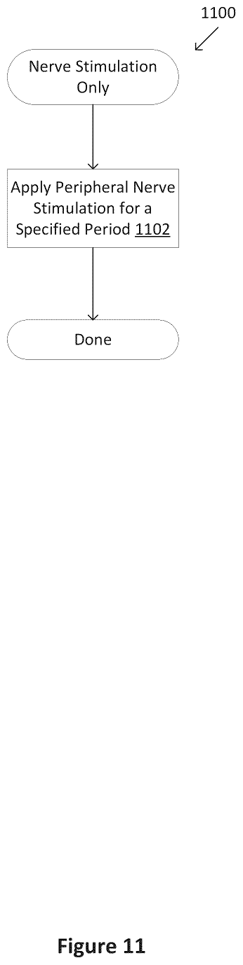

In other exemplary embodiments, only peripheral nerve stimulation is applied to the subject to treat the subject to enhance performance by the subject. FIG. 11 depicts a flowchart 1100 for some embodiments. At 1102, the peripheral nerve stimulation is applied via a nerve stimulator, like 806, as a current of the specified amperage at the specified random frequencies for the specified durations identified above relative to FIG. 7. The sessions may be repeated as detailed above relative to FIG. 9

The particulars of the treatment may depend on the goal of the treatment. The protocols may vary as to duration, frequency, and/or current level of stimulation. For example, for enhancing relaxation or increasing calm in a patient, the treatment may entail MNS with guided meditation, mental imagery, and/or guided deep breathing. The MNS may, for example apply a current of 2-4 mAmp at a frequency of 4-15 Hz for 5 minutes. For improved motor learning and performance as to reaction time and motor coordination, MNS may be applied during training for the particular activity or sport. The MNS stimulation may be 30 minutes in duration and with a current of 2-4 mAmp and a frequency of 20-50 Hz.

A study of a training approach like that described above was performed. The study was an exploratory randomized, double-blinded, sham-controlled, two-arm parallel-group clinical trial. The study was approved by the Bioethics and Research Committees from Universidad Autonoma de Aguascalientes (Aguascalientes, Mexico). Written informed consent from each participant's parent or legal guardian was obtained; all participants assented to participate. Participants were randomly assigned with a 50% chance of being allocated to either of the two groups. Both groups were exposed to ten training sessions of 30 minutes each. Participants were evaluated at three-time points: pre-intervention, 130 post-intervention, and 1-month follow-up.

Study subjects were recruited from local pediatric clinics and referrals from pediatric centers at the Neuromodulation Center NEOCEMOD in Aguascalientes City, Mexico. A total of 60 participants were enrolled in the study. Demographics and medications of the population are detailed in Table 1. The inclusion criteria were: (1) ADHD diagnosis performed by a board-certified clinician according to the American Psychiatric Association's Diagnostic and Statistical Manual, Fifth edition (DSM-5) (American Psychiatric Association, 2013) and the International Classification of Diseases, Tenth Revision (ICD-10) (World Health Organization, 1992); (2) between 8 to 18 years of age; and (3) on stable medication doses for at least 3 months previous to enrollment. The exclusion criteria included: (1) comorbidity with severe neurological or psychiatric disorder; (2) comorbidity with uncontrolled chronic medical diseases such as diabetes, cardiopathies, or renal failure; (3) any other medical condition that in the view of the investigator could affect the participation of the subject.

ADHD symptoms severity including behavioral problems, hyperactivity-impulsivity, learning problems, anxiety, and psychosomatic symptoms, were assessed using the revised Conners' Parent Rating Scale (CPRS-R). The CPRS-R is a popular research and clinical tool for obtaining parental reports of childhood behavior problems.

Awake EEG recordings were recorded at rest in closed-eyes condition, each recording lasting 5 minutes. EEGs were recorded according to the American Clinical Neurophysiology Society recommendations using the 10/20 International System, at a sample rate of 500 Hz, amplified and filtered using a bandpass of 0.3-50 Hz using the EEG-amplifier Neuroamp II (BEE medic, Switzerland). The EEG assessment allows for analyzing the spontaneous cortical activity of the brain. This activity provides information about the functional state of endogenous brain oscillations and has been widely used to assess ADHD. Multiple biomarkers have been proposed to study this pathology and may provide objective measurements to support the clinical outcomes of the study.

Participants received a total of 10 sessions through 5 consecutive days per week over a two-week period. Each intervention session had two stages: 1) 5 minutes of R-BFB-paced respiration training with MNS; followed by 2) 20 minutes of sensorimotor rhythm NFB (SMR-NFB) training with MNS. Both groups were exposed to verum R-BEN and SMR-NFB, however, the active group (AG) received active MNS, whereas the sham group (SG) received sham MNS. R-BFB and SMR-NFB were delivered using the ProComp Infiniti Encoder, an 8-channel, battery-powered system for real-time physiological data acquisition.

The R-BFB training was designed to induce relaxation in the subjects. R-BFB was individualized to each participant after a 5-min baseline recording of respiratory and heart rates. Mean respiratory rate was obtained, and a therapist guided each participant to slow down breathing through deep inhalations, as tolerated by the participant. Special attention was placed on slow diaphragmatic breathing. Graphic feedback via computer animations and coaching from the therapist were provided to each participant throughout the R-BFB sessions.

EEG signal for the NFB sessions was recorded using an active recording electrode placed at Cz with reference to linked earlobes. The pre-set feedback parameters were as follows (Morales-Quezada et al., 2019): theta inhibition (4-7 Hz), SMR reinforcement (12-15 Hz) and inhibition of high beta (25-35 Hz). The NFB-SMR training sessions consisted of 5 trials of 3 minutes each, inter-trial interval of 30 seconds, and the whole SMR NFB session lasted approximately 20 minutes. The visual display for participants included a puzzle with three bars representing each frequency band. One piece of the puzzle was open, and bars turned green whenever the participant achieved the parameters for 0.5 seconds, indicating a positive reward. This was reinforced by an auditory stimulus presented as a bell. In addition, by opening subsequent puzzles the participant could see a numerical reward of the points earned.

MNS was delivered at a maximum of 2 mA of current for the duration of each of the R-BFB (5 minutes) and SMR-NFB (20 minutes) training sessions. These parameters were selected based on most stimulation characteristics using electrical stimulation with weak currents, and focusing on promoting EEG entrainment. Thus, MNS was delivered at a random frequency between 4-10 Hz during R-BFB to promote a state of relaxation, while a randomly oscillating frequency delivered between 12-16 Hz were used to facilitate SMR entrainment during NFB. For the sham condition, stimulation was applied for a period of 30 seconds and then the device turned off automatically.

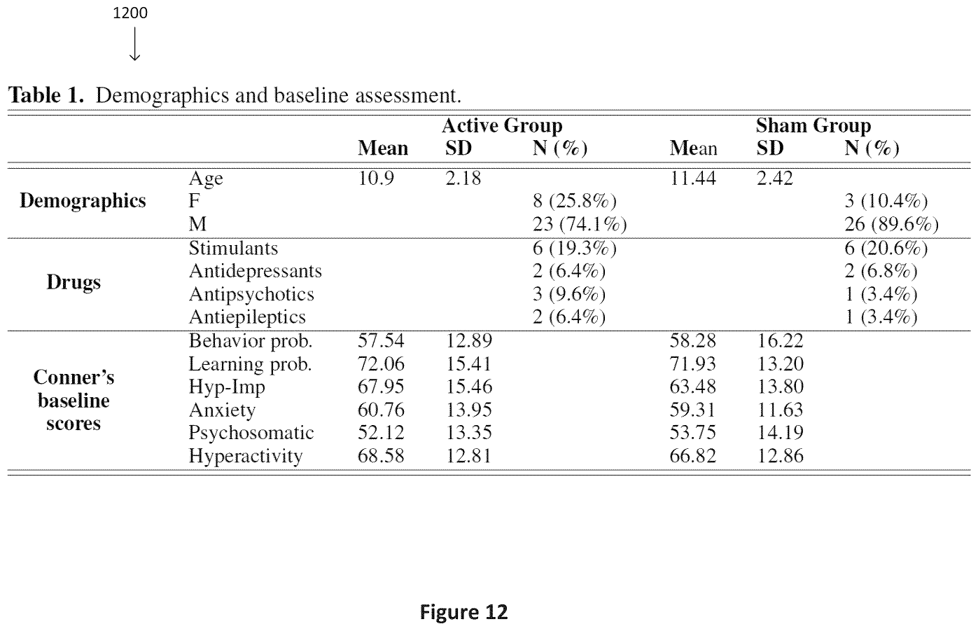

A total of 60 children with ADHD diagnoses were enrolled and randomized to participate in this trial to receive active MNS and R-BFB/NFB (AG) and sham MNS and R-BFB/NFB (SG). All participants tolerated the interventions well, and no direct side effects associated with any of the treatment arms were reported. The table 1200 in FIG. 12 details baseline, demographic, and clinical characteristics.

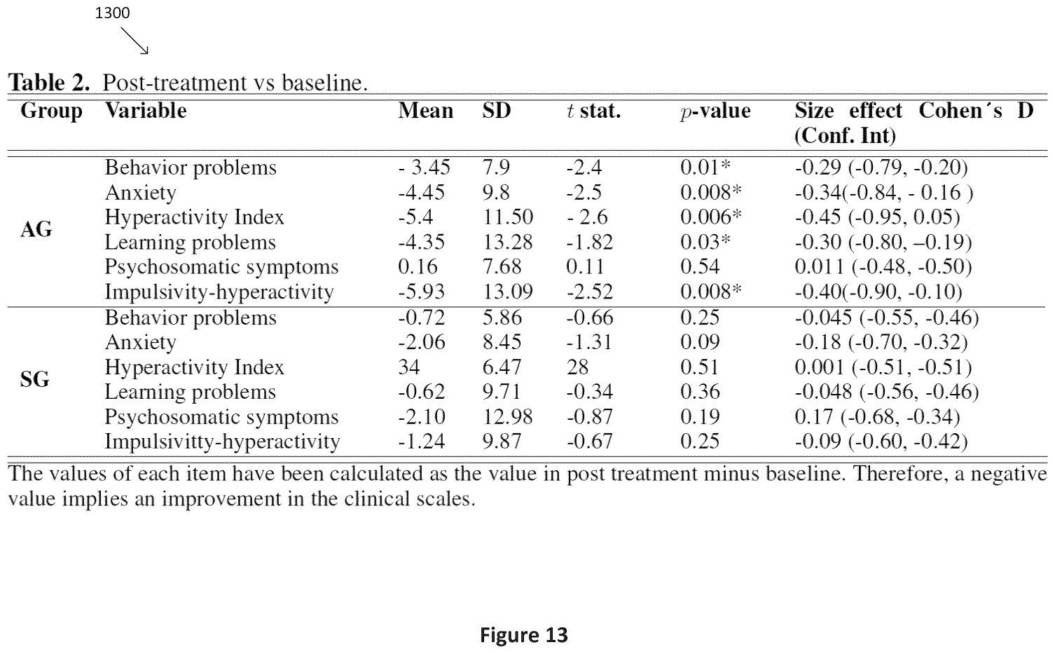

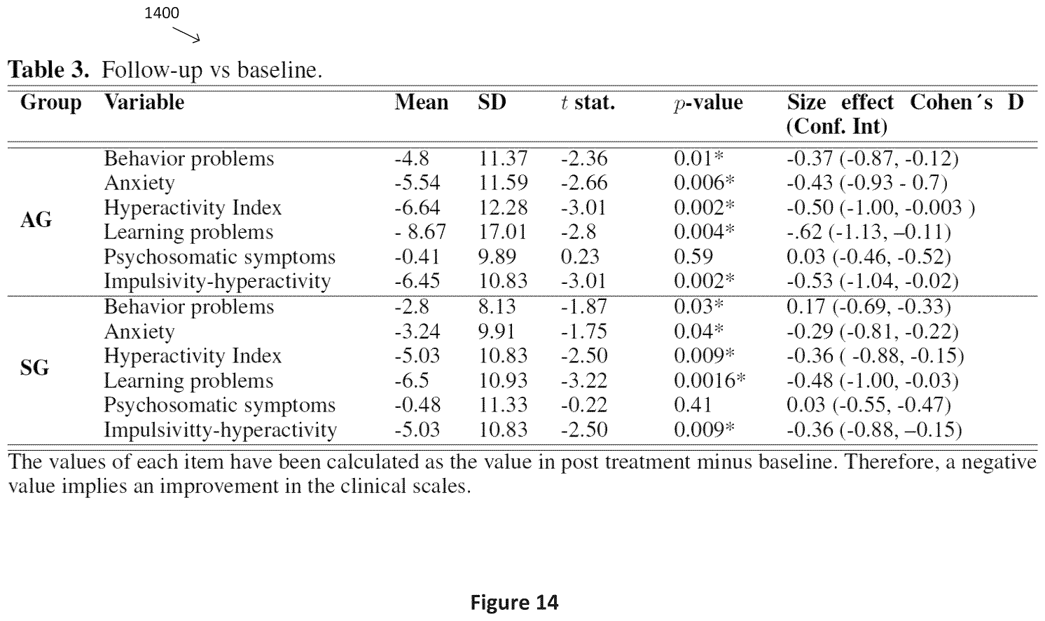

FIGS. 13 and 14 show tables 1300 and 1400 containing the results of paired t-tests conducted within the groups to compare post-treatment (table 1300) and follow-up scores with baseline scores (table 1400). In the AG group, there was a significant improvement observed in several instrument categories, such as behavior problems, anxiety, hyperactivity Index, learning problems, and impulsivity-hyperactivity, both immediately after the intervention and at follow-up. These improvements had small to medium-sized effects. On the other hand, the SG group showed significant symptom improvement only at follow-up, not immediately after the intervention. However, when comparing the AG and SG groups using paired t-tests, no statistically significant differences were found in any of the Conner's ADHD subcategories.

Multiple linear regression models were used to explore further whether the intervention, age, gender, and medication significantly predicted the main clinical outcomes of Conner's subscales. A significant interaction between the treatment group and clinical outcomes was not found at any time point.

After this, models were constructed to evaluate the effect of Conner's subscales without considering the group treatment allocation, at both evaluation time points (after intervention and at follow-up) and the dependent variables of age, gender, and if the participants were taking medication. The hyperactivity index overall regression model was statistically significant (R2=0.06, F-value=2.5, p-value=0.03). It was found that the evaluation at follow-up improved when compared to baseline scores [(β=−5.8], p-value=0.01). Males showed significantly less overall improvement when compared to females [(β=5.24], p-value=0.03), while age and medication did not significantly predict the hyperactivity index scores at any of the evaluation time points.

The learning problems model was overall statistically significant (R2=0.10, F-value=3.97, p-value<=0.002). Both intervention groups significantly improved at follow-up when compared to baseline scores [(β=−7.65], p-value=0.002). Age was also significant, [(β=−1.20], p-value=0.006) indicating that older children improved more than the younger in treatment group.

Statistically significant changes over time were not found for behavioral problems, anxiety, psychosomatic symptoms, or impulsivity-hyperactivity Conner's subscales.

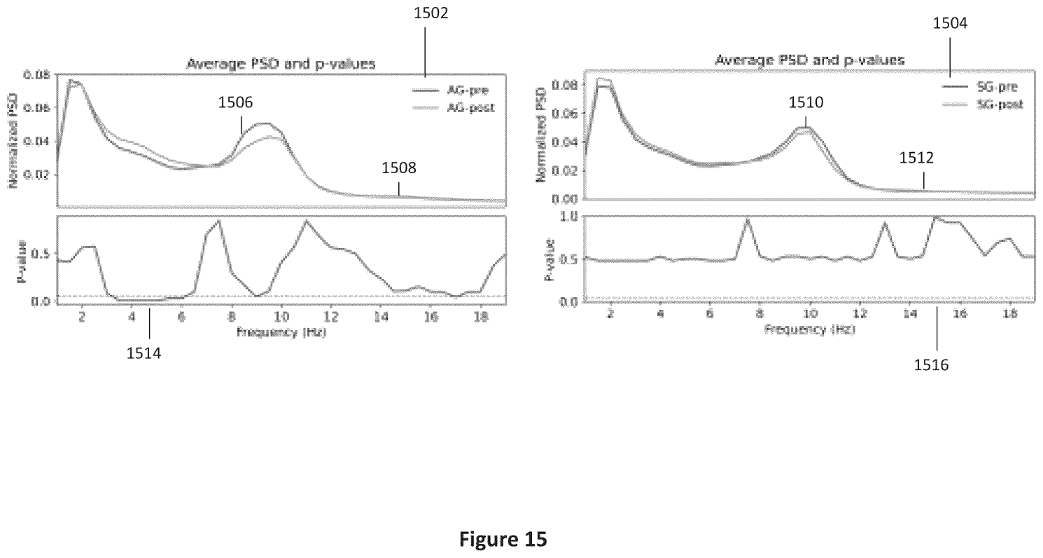

The EEG analysis also provided some interesting results. FIG. 15 shows plots the averaged PSD of the basal EEG recordings over subjects and channels for AG (1502) and SG (1504) in the baseline (see 1506 and 1510) and post-treatment (see 1508 and 1512) evaluation sessions under the closed-eyes condition. The Wilcoxon Signed Rank Test was applied to find statistical significant changes in PSD values for each frequency point. The false discovery rate (FDR) was corrected following the Benjamini/Hochberg approach. As can be seen, the power distribution of the AG (see plot 1514) shifted towards slower frequencies after the intervention. The power of the Theta band increased, reaching statistical significance in the range from 3.5 to 6 Hz (p-value<0.05). On the other hand, the power of Alpha decreased, reaching statistical significance (p-value<0.05) around 9 Hz. These results were not observed in the SG (see plot 1516). In this case, no statistically significant changes are detected in the PSD of the EEG between baseline and post-evaluation sessions.

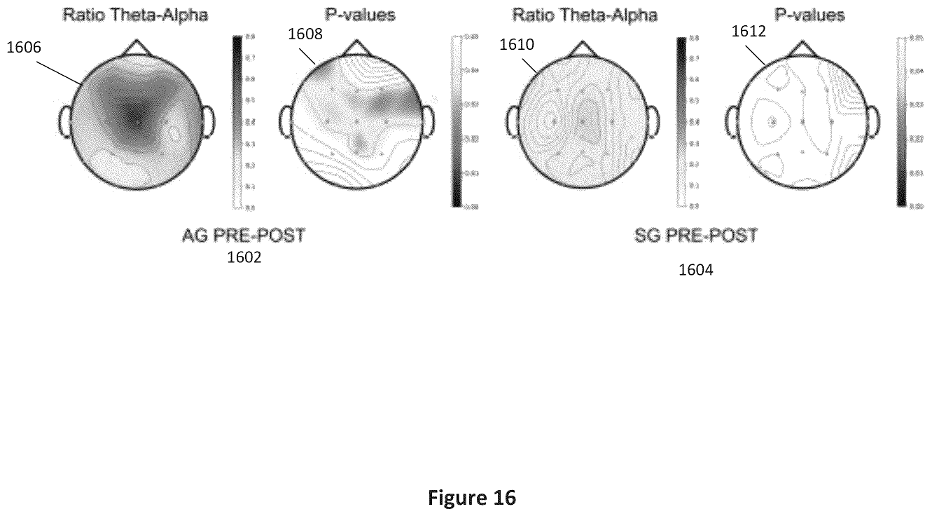

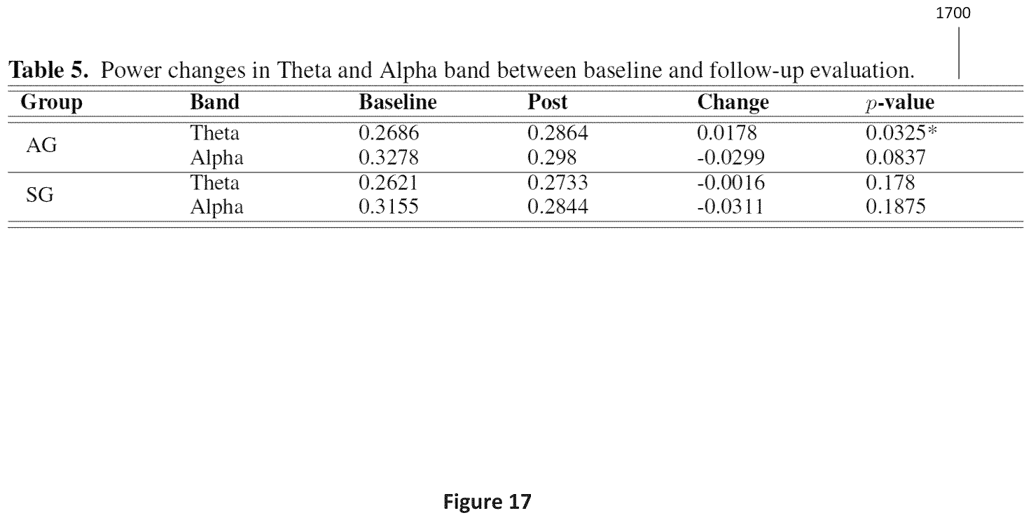

FIG. 16 shows the scalp distribution of the change between baseline (1606) and post-treatment (1610) evaluation sessions of the Theta to Alpha ratio (TAR) for AG and SG. As before, Wilcoxon Signed Rank Test was applied to calculate the p-values (see 1608 and 1612), including FDR correction with the Benjamini/Hochberg approach. As can be observed, the TAR was significantly increased, especially in central electrodes (i.e., F3, FZ, F4, C3, CZ, C4, P3, PZ, P4). In order to study the relationship between Theta and Alpha in these areas with more detail, FIG. 17 depicts table 1700 that provides the power in these bands averaged across the central electrodes for both groups in pre-evaluation and post-evaluation sessions. These results show a statistically significant increase of Theta power while Alpha decreases, confirming the findings of FIG. 16. In the case of the TBR, no statistical differences between groups or between baseline and post-evaluation sessions were found.

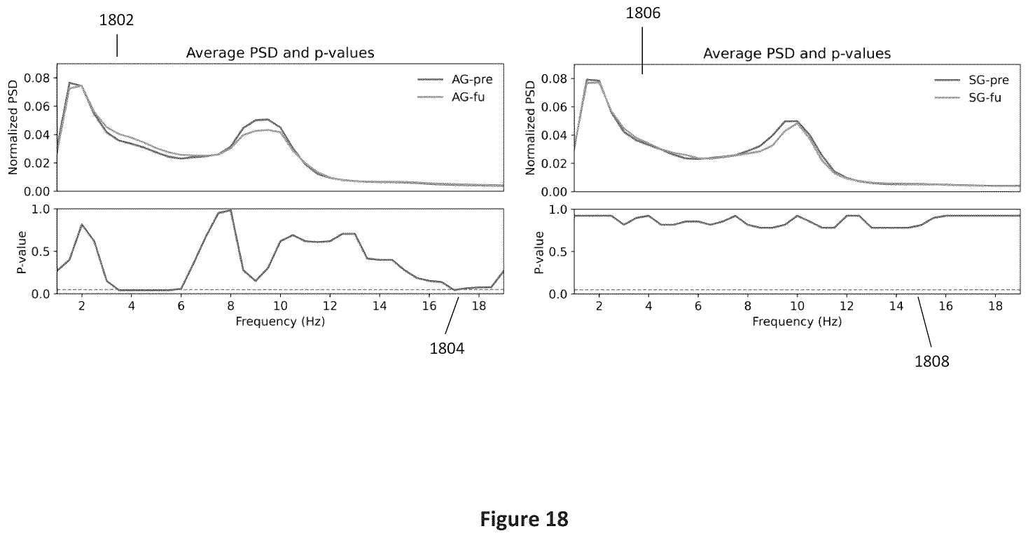

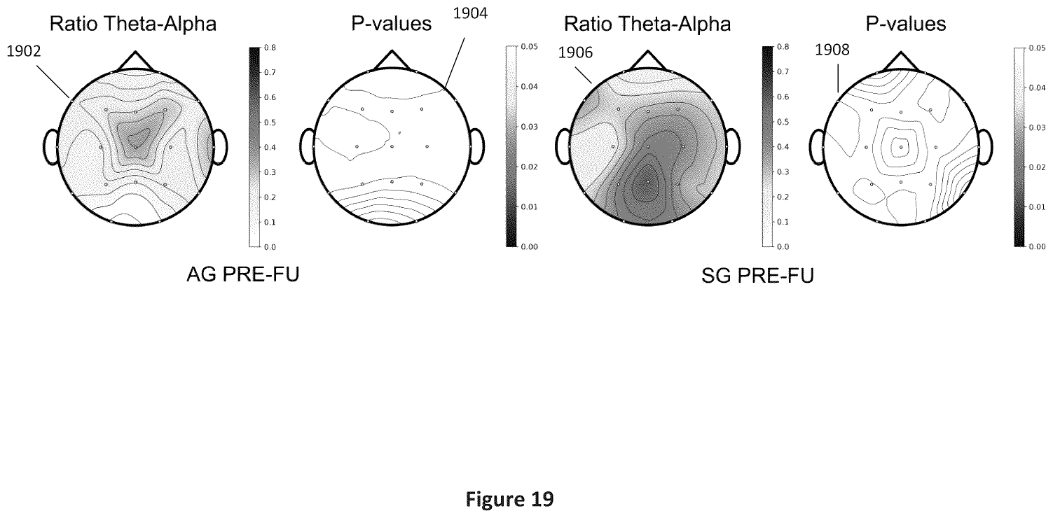

The same analysis was performed to study the changes between baseline and follow-up sessions. As can be seen in plots 1802, 1804, 1806, and 1808 in FIG. 18 and in scalp distribution 1902, 1904, 1906, and 1908 in FIG. 19, the changes that were appreciated between baseline and post-evaluation sessions are mainly maintained, although their statistical significance has decreased. The same applies to the power in Theta and Alpha bands, which now is only statistically significant for the power increase in Theta, as shown in the table of FIG. 17.

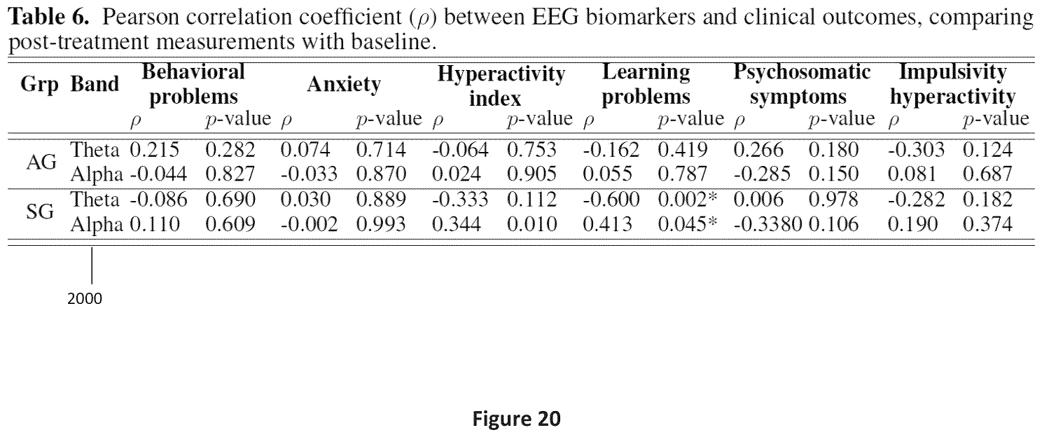

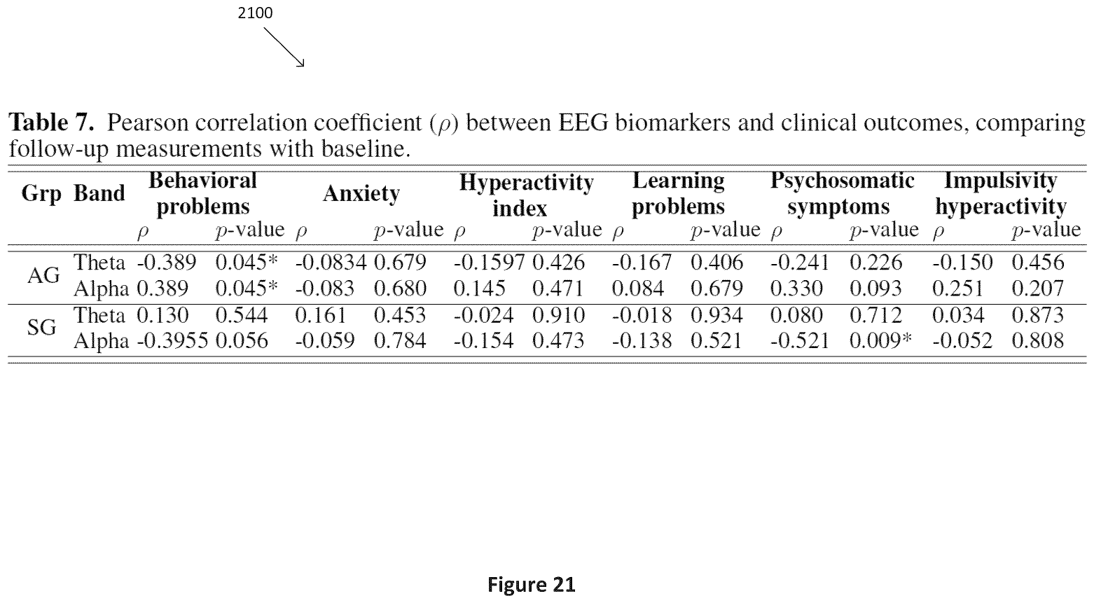

Regarding the correlation analysis, Table 2000 of FIG. 20 and table 2100 of FIG. 21 show the correlation of the power changes in Theta and Alpha bands with the clinical changes for each variable in AG and SG, calculated with Pearson's method. The p-values were calculated with a hypothesis test whose null hypothesis is that the two sets of input data were uncorrelated. For this analysis, we only used the central channels (i.e., F3, FZ, F4, C3, CZ, C4, P3, PZ, P4) for two reasons: the subjects received MNS and SMR-NFB in this area, and it is where the changes of TAR were more strong (see FIGS. 16 and 19).

Regarding the clinical outcomes of the R-BFB/NFB intervention, the children of the AG showed significant improvement in the post-treatment evaluation session when compared to baseline in the following categories: behavioral problems, anxiety, hyperactivity index, and impulsivity-hyperactivity. On the other hand, the SG group did not show any statistically significant improvement at the end of the intervention. Regarding the follow-up evaluation, both groups showed significant improvements on all Conner's subscales but psychosomatic symptoms. Nevertheless, the mean improvement is larger in the AG than in the SG in most categories, as demonstrated by having higher effect size when compared to the SG. These findings suggest that the application of MNS to boost the R-BFB/NFB therapy is more effective in treating ADHD symptoms compared to the protocol without stimulation. In this regard, it is worth to mention that the SG received sham stimulation to control for placebo responses. Yet, the results at follow-up demonstrates the powerful role of treatment expectations associated with technology-based interventions, and the placebo by proxy effect observed in the SG.

The results of the EEG analyses support these findings with objective biomarkers, showing significant alterations in the spontaneous brain activity after the intervention for the AG, but not for the SG. Thus, the shift of the EEG to slower frequencies may be related to the improvement in behavior problems, anxiety and impulsivity-hyperactivity indices reported by the clinical assessment in AG.

While exemplary embodiments have been described herein, various changes in form and detail may be made without departing from the intended scope of the invention as defined by the appended claims.

1. A method of treating a subject to enhance performance by the subject, comprising:
initiating a session of peripheral nerve stimulation with a subject by applying electrical stimulation to a peripheral nerve of the subject to enhance the performance by the subject; and
randomly varying frequency, pulse width, duration, or pulse amplitude of the electrical stimulation during at least a portion of the session.

2. The method of claim 1, wherein more than one of frequency, pulse width, duration and pulse amplitude of the electrical stimulation are randomly varied.

3. The method of claim 2, wherein the more than one of the frequency, pulse width, duration and pulse amplitude are randomly varied within specified ranges during some of the session.

4. The method of claim 1, wherein the frequency, pulse width, duration or the pulse amplitude of the electrical stimulation are varied during multiple portions of the session.

5. A method of treating a to enhance performance by a subject, comprising:
with a biometric sensor, measuring a biometric of the subject;
providing biometric feedback to the subject based on the measured biometric; and
applying peripheral nerve stimulation via a peripheral nerve stimulator to the subject as the biometric feedback is provided to the subject for a specified duration to enhance the performance by the subject, wherein a frequency, a pulse width, a duration, or a pulse amplitude of the stimulation randomly varies during at least a portion of the applying.

6. The method of claim 5, wherein the biometric sensor is a respiration sensor.

7. The method of claim 6, wherein the biofeedback comprises an indication of respiration rate and/or respiration pattern or an indication of brain activity of regions of a brain of the subject.

8. The method of claim 7, wherein a representation of the respiration rate and/or respiration pattern is displayed on a display of a computing device as part of the biofeedback.

9. The method of claim 5, wherein the peripheral nerve stimulator is a median nerve stimulator.

10. The method of claim 5, wherein the peripheral nerve stimulator applies an electrical stimulus to the subject.

11. The method of claim 10, wherein the electrical stimulus applies a current between 0.25 mA and 10 mA to the subject.

12. The method of claim 10, wherein the electrical stimulus has a frequency in the range between 0.25 Hz and 30 KHz.

13. The method of claim 10, wherein the electrical stimulus is applied for a duration in a range between 1 minute and 50 minutes.

14. A method of treating a subject to enhance performance by the subject, comprising:
conducting respiratory biofeedback (R-BFB)-paced respiration training with the subject;
providing median nerve stimulation to the subject via a median nerve stimulator during the R-BFB-paced respiration training to enhance performance by the subject;
conducting sensorimotor rhythm neurofeedback (SMR NFB) training with the subject;
providing median nerve stimulation to the subject via a median nerve stimulator during the SMR NFB training to enhance the performance by the subject; and
wherein a frequency of the stimulation, a pulse width of the stimulation, a duration of the stimulation, or a pulse amplitude of the stimulation randomly varies during at least a portion of at least one of the providing of the median nerve stimulation during the R-BFB paced respiration training or the providing of the peripheral nerve stimulation during the SMR NFB training.

15. The method of claim 14, wherein the median nerve stimulator provides an electrical stimulus.

16. The method of claim 14, wherein the R-BFB-paced respiration training is conducted with the subject for a duration between 1 to 10 minutes.

17. The method of claim 14, wherein the SMR NFB training is conducted with the subject for a duration between 5 and 50 minutes.

18. The method of claim 14, wherein the R-BFB-paced respiration training comprises providing the subject with feedback regarding a respiration rate of the subject or a respiration pattern of the subject via a computing device.

19. The method of claim 14, wherein the SMR NFB training comprises providing the subject with biofeedback regarding activity of certain brain regions of the subject via a computing device.

20. A system for treating a subject to enhance performance by the subject, comprising:
a biometric sensor for sensing a biometric of a subject;
a computing device, comprising:
a storage storing computer programming instructions;
a processor configured for executing the computer programming instructions to cause the processor to provide biofeedback training to the subject using the sensed biometric from the biometric sensor; and
a peripheral nerve stimulator configured to stimulate a peripheral nerve of a subject during the biometric training to enhance the performance by the subject, wherein a frequency, a pulse width, a duration, or a pulse amplitude of the stimulation randomly varies during the applying.

21. The system of claim 20, wherein the biometric sensor comprises a respiration sensor that senses a respiration rate and/or a respiration pattern of the subject.

22. The system of claim 20, wherein the biometric sensor comprises an electroencephalogram (EEG).

23. The system of claim 20, wherein the peripheral nerve stimulator is a median nerve stimulator that provides an electrical stimulus.

24. The system of claim 20, wherein the biofeedback training is respiratory biofeedback training or sensorimotor rhythm biofeedback training.

25. The system of claim 20, wherein the pulse width of the stimulation randomly varies during the applying.

26. A method of treating a subject to enhance performance by the subject, comprising:
engaging a subject in a form of treatment;
initiating a session of peripheral nerve stimulation with the subject to enhance performance by the subject in conjunction with the treatment by applying electrical stimulation to a peripheral nerve of the subject; and
randomly varying frequency, pulse width, duration, or pulse amplitude of the electrical stimulation during at least a portion of the session.

27. The method of claim 26, wherein the treatment is one of biofeedback training, neurofeedback training, administering medication to the subject, playing a game with the subject, having the subject interact with a computer program, engaging with a therapist, or meditation.
